(19) United States
(12) Patent Application Publication
(10) Pub. No.: US 2017/0209405 A1
(43) Pub. Date: Jul. 27, 2017

(54) APPLICATION OF COMPOUND MIXTURES TO CONTROL OXIDATION

(71) Applicant: Abraham Fouad JALBOUT, Hermosillo (MX)

(72) Inventor: Abraham Fouad JALBOUT, Hermosillo (MX)

(21) Appl. No.: 15/354,794

(22) Filed: Nov. 17, 2016

Related U.S. Application Data
(60) Provisional application No. 62/281,672, filed on Jan. 21, 2016.

(51) Int. Cl. A61K 31/225; A61K 31/375; A61K 31/593; A61K 31/355; A61K 31/015; A61K 45/06; A61K 33/04; A61K 31/198; A61K 31/20; A61K 36/258; A61K 31/465; A61K 31/185; A61K 31/385; A61K 31/12

(52) U.S. Cl. CPC A61K 31/225; A61K 31/385; A61K 31/375; A61K 31/593; A61K 31/355; A61K 31/015; A61K 31/12; A61K 33/04; A61K 31/198; A61K 31/20; A61K 36/258; A61K 31/465; A61K 31/185; A61K 45/06

(57) ABSTRACT

A combination of antioxidants is proposed as a supplement based on the best available clinical data. The present invention proposes a research-driven approach premised on the basic tenet that there are multiple antioxidant systems and various rate limiting steps. According to the present invention, specific combinations can influence the oxidative process in a way that cannot be achieved by any one individual molecule. It is an object of the present invention to develop and evaluate various combinations of antioxidants in order to determine which combinations are most effective in the treatment of specific conditions. It is a further object of the present invention to use clinical data to determine which combinations of antioxidants consistently yield clinically viable results.

### CROSS REFERENCE TO RELATED APPLICATIONS

This application claims priority to U.S. Provisional Patent Application No. 62/281,672 filed Jan. 21, 2016, which is hereby incorporated by reference in its entirety.

### BACKGROUND OF THE INVENTION

Field of the invention

This invention relates to a method of mitigating oxidative stress and treating a variety of conditions and ailments. More particularly, this invention discloses a novel approach to the administration of an antioxidant regimen that supports a multitude of enzymatic pathways. The antioxidant cocktail herein described overcomes the difficulties of oxidative stress and can be used to prevent or treat a disorder which has a component of oxidative stress or to maintain, optimize or boost a subject's overall immunological response.

### Description of Related Art

### Oxidation and Oxidative Stress in Biological Systems

Oxidation is a chemical reaction whereby the transfer of electrons (hydrogen) from a substance to an oxidizing agent takes place. For example, metals such as iron and copper are capable of donating one or more electrons to oxygen in the atmosphere (i.e., “rusting”) to form metal oxide compounds. In biological systems, oxidative stress (i.e., “biological rusting”) occurs in the presence of a reactive oxygen species that goes beyond the body's natural ability to protect cells from attack by oxygen. An imbalance in the ability of individual cells to neutralize the oxidative potential will result in damage to cellular components. When there are disruptions in the natural oxidation processes, highly unstable and potentially damaging molecules (free radicals and/or peroxides) can be created. This occurs through actions on lipids, proteins and DNA oxidative components.

Free radicals are a type of highly reactive metabolite that is naturally produced by the body as a result of normal metabolism and energy production when certain molecules interact with oxygen. Free radicals are a natural biological response to environmental toxins such as cigarette smoke, sunlight, chemicals, cosmic and manmade radiation, and are even a key feature of sonic pharmaceutical drugs. Free radicals are also produced during exercise, and wherever inflammation occurs in the body. Once formed, free radicals can start a chain of damaging chemical reactions—aggressively attacking other molecules in the body in order to capture missing electrons in a reaction known as oxidation.

Free radicals are inherent in the aerobic metabolism of living organisms and are generated by both physiological and pathological processes. They are sometimes generated intentionally to serve biological functions, such as microbicides in phagocyte cells, or may be accidents of chemistry following which they exhibit destructive behaviors. Whatever their mechanism of generation, if free radical production and removal is not controlled, then their effects on an organism can be damaging. To combat excessive and inappropriate damage, an elaborate system of antioxidant defenses has evolved.

### Medical Effects of Oxidative Stress

When there is an unbalance between the oxidants and the antioxidants in favour of the oxidants, a condition of oxidative stress exists that can lead to tissue damage. Because free radicals contain an unstable number of electrons, free-radical oxidation has a cascading effect in biological systems, whereby a tree, radical reacts with an otherwise healthy molecule (such as DNA and cell membranes), and creates a new free radical which continues the oxidation reaction. For example, free radicals can trap a low-density lipoprotein (LDL) in an artery wall and begin the formation of plaque; they can damage DNA; or they can change the course of what enters and leaves a cell. Oxidative stress is thought to contribute to the development and exacerbation of many of the modern day major diseases. These range from Alzheimer's disease, schizophrenia, bipolar disorder, stroke and Parkinson's disease to atherosclerosis including heart failure and myocardial infarction. There is emerging evidence that oxidative stress is involved in the pathogenesis of fatty liver (non-alcoholic steatohepatitis, NASH), obesity and type-2 diabetes.

Oxidative stress has also been implicated in chronic fatigue syndrome and proposed to be a contributor to the aging process (Harman's free radical theory of aging) and this notion has been supported by work on D. melanogaster and C. elegans. Short-term oxidative stress may be important in prevention of aging by induction of a process named mitohormesis. Oxidative stress is thought to be involved in age-related development of cancer. The reactive species produced in oxidative stress can cause direct damage to proteins, fats, carbohydrates, and DNA. Additionally, they may harm cells that line blood vessels, permitting tumor cells to enter the bloodstream and metastasize.

The human body naturally produces many different types of molecules called antioxidants to combat free radicals and protect the cells from attack by oxygen. Antioxidants can safely interact with free radicals and stop the chain of damaging reactions before damage is done to cells. There are several enzyme systems in the body that scavenge for and “quench” free radicals, and many vitamins ingested in foodstuffs exhibit antioxidant activity, such as vitamins C and F, along with mineral antioxidants such as selenium and manganese, and plant compounds that act as antioxidants such as beta-carotene and lycopene.

As the name suggests, an antioxidant is a molecule that inhibits the oxidation of other molecules by removing free radical intermediates. As electron donors, antioxidants can break the chain reaction of free radicals by sacrificing their own electrons to oxidize free radicals, without becoming free radicals themselves; thus, by being oxidized themselves, antioxidants often act as reducing agents in biological systems. Antioxidants are nature's way of providing cells with an adequate defense against attack by reactive oxygen species (ROS).

In a given biological system, there is vast array of antioxidant activity characterized by a multitude of enzymatic pathways, and it is not well understood precisely how all of these different systems work together to protect the body from free-radical damage. However, it is generally recognized that no one antioxidant can provide the protection offered by the many antioxidants working together in combination. A few well-known antioxidant enzyme pathways involving glutathioneperoxidase, superoxide dismutase (SOD) and catalase have been examined in the literature. However, other enzymatic antioxidants that are less well-studied may emerge as equally important factors in due course. Other enzymes such as aldehyde dehydrogenases also exhibit antioxidant properties, although not in a primary role.

Oxidation is the transfer of electrons (hydrogen) from a substance to an oxidizing agent. Metals such as iron and copper are capable of accepting or donating a single electron. The most important of these reactions is Fenton's reaction in which a hydroxyl radical is produced from reduced iron and hydrogen peroxide. An imbalance in the ability of an individual cell to neutralize the oxidative potential will result in damage to cellular components. The presence of such metals in biological systems is essential but also implicated in the level of oxidative stress. Lesser degrees of stress can be easily overcome and the cell can regain its original state, whereas greater degrees of oxidative stress can initiate apoptosis, while intense stresses may cause cell death and tissue necrosis.

### Clinical Practice

Antioxidants are administered—both by physicians and as self-medication by laypersons—as individual agents or in combination as part of a pre-determined regimen. The underlying mechanism of antioxidant activity varies depending on the molecule (reagent), and a treatment regimen can therefore be modified according to different activities of different molecules. Some molecules are precursors which will be converted to downstream antioxidants; the clearest example being methionine or N-acetyl cysteine, which is metabolized in order to replenish stores of glutathione when treating patients with acute liver failure. Other antioxidants may be co-factors or catalysts for biological processes, and still others that may have a direct anti-oxidative effect as reducing agents.

When classified according to their solubility, antioxidants can be categorized as either soluble in lipids/fat (hydrophobic) or water (hydrophilic). The latter react with oxidants in the cell cytosol and the blood plasma, while antioxidants protect cell membranes from lipid peroxidation. Because free radicals can attack either the watery cell contents or the fatty cellular membrane, the body requires both types of antioxidants to ensure full protection from oxidative damage.

Solubility is not the only way to categorize antioxidants. They can also be categorized as enzymatic and non-enzymatic antioxidants. Enzymatic antioxidants break down and remove free radicals. They can also flush out dangerous oxidative products by converting them into hydrogen peroxide, and subsequently into water through a multi-step process that requires a number of trace metal cofactors, such as zinc, copper, manganese, and iron. Enzymatic antioxidants cannot be found in supplements, but instead are produced in the body.
The main enzymatic antioxidants are:
   Superoxide dismutase (SOD) can break down a superoxide into hydrogen peroxide and oxygen, with the help of copper, zinc, manganese, and iron. It is found in almost all aerobic cells and extracellular fluids.
   Catalase (CAT) works by converting hydrogen peroxide into water and oxygen, using iron and manganese cofactors. It completes the detoxification process started by SOD.
   Glutathione peroxidase (GSHpx) and glutathione reductase are selenium-containing enzymes that help break down hydrogen peroxide and organic peroxides into alcohols. They are most abundant in the liver.
Non-enzymatic antioxidants, on the other hand, function by interrupting free radical chain reactions. Some examples are carotenoids, vitamin C, vitamin E, plant polyphenols, and glutathione (GSH). Most antioxidants found in supplements and foods are non-enzymatic, and they provide support to enzymatic antioxidants by doing a “first sweep” and disarming the free radicals. This helps prevent enzymatic antioxidants from being depleted.

There is some available data on the usefulness of antioxidants, particularly as it relates to vitamin supplementation. However, there is a marked lack of current research data on selection and administration of various antioxidant compounds in combination. This scarcity of research data is compounded by the existence of conflicting results in the available data that has been reported. For example, although vitamin E has been shown to have a beneficial effect in some studies of liver and heart disease for Alzheimer's patients, the administration of vitamin E as a dietary supplement has mixed results in the available literature. In addition, beta-carotene has been shown to decrease cognitive decline due to oxidative stress, but for smokers high doses of beta carotene has been shown to increase the rate of lung cancer. Yet in spite of such contradictions, antioxidants remain one of the most promising avenues for future research, and a number of pharmaceutical endeavors are focused on the development of specific antioxidant treatment regimens, such as the use of radical-scavenging nitrones as neuroprotective agents in the treatment of strokes.

Antioxidants: Drug vs. Supplement

The potential value of antioxidants for the treatment of chronic disease and/or maintenance of general health is becoming increasingly recognized. However, there is no apparent consensus regarding the use of individual chemicals as a drug or supplement versus a the administration of a combination of compounds derived from naturally-occurring plant based substances. It is likely that a combination of antioxidant agents will prove most effective in pharmaceutical treatment applications, as is the case for treatment of chronic kidney disease. Furthermore, pharmacological doses of one antioxidant may lead to unwanted downstream effects (e.g., administration of methionine without adequate amounts of folic acid, vitamin B6 and B12 will lead to the production of homocysteine). For general health, it is therefore recommended to administer a combination of naturally occurring substances that likely will function as an adjunct to a healthy diet and reduce or compensate for the potential deficiencies or unwanted side effects caused by other antioxidants administered as part of the same treatment regimen. Incidentally, these issues likely account for some of the variability in the research findings that have occurred in the area of antioxidants.

There has long been a demand for antioxidants among food manufacturers, especially as research efforts continue to suggest that some antioxidants are better than others at quenching free radicals. Beta carotene, for instance, combats superoxide dismutase, but vitamin E has little effect against it. Alternatively,vitamin E makes LDL more resistant to oxidation. The oxidized LDL will likely form plaque deposits and clog arterial walls. The functions of these antioxidants opens the door to a marketing specialist for supplement sales.

Enzymatic functions such as those described above allow antioxidant supplements to be of potential use in the world market and there is an increasing necessity for such products, including:
Repairing damaged molecules—Some unique types of antioxidants can repair damaged molecules by donating a hydrogen atom. This is very important when the molecule is a critical one, such as DNA.
Blocking metal radical production—Some antioxidants have a chelating effect—they can capture toxic metals like mercury and arsenic, which can cause free radical formation, and prevent any chemical reaction from taking place. Water-soluble chelating agents can also help to excrete toxic metals out of the body through the body's urine.
Stimulating gene expression and endogenous antioxidant production—Some antioxidants can stimulate the body's genes and increase its natural defenses.
Providing a “shield effect”—Antioxidants, such as flavonoids, can act as a virtual shield by attaching to DNA to protect it from free radical attacks.
Promoting cancer cells to “commit suicide”—Some antioxidants can provide anti-cancer chemicals that halt cancer growth and force some cancer cells to self-destruct (apoptosis)

Most importantly, the market for research-driven antioxidant supplements is large and continually expanding. Concrete, immediately addressable markets include:
Government agencies in foreign countries such as Mexico or China which is in the hundreds of millions USD and growing. For these medications such countries are using the public health system (through government support) to implement distribution.
Health Stores have hundreds of vitamin supplements all of which are speculated to work as antioxidants but do not have the scientific backing to support such claims.
Multi-Level Sales are becoming increasingly competitive and unique in their business model. They are targeting sales from external sales personnel from a network of users.

In short, demand for antioxidants is growing to a capacity that is far from being saturated. Based on the Food Marketing Institute's 2011 “Shopping for Health” survey, antioxidants are among the principal health components that US consumers strive for in food products. While this tendency has been picking up over the last few years, buyers seek results oriented research as evidence for the success of these products.

As such, it is important for consumers to understand antioxidants and how they may assist in their general well-being. Based on a 2010 Gallup Study of Nutrient Knowledge & Composition, 32% of women make a strong effort to consume foods/beverages rich in antioxidants, compared to 24% of men. The worldwide volume of antioxidants in 2007 was 0.88 million tons with a value of 3.7 billion USD. This represents an annual rate of growth of 3.9% with an expected 1.25 million tons to be produced by the year 2016. The Mexican and Chinese markets have a speculated two-fold increase in market capacity.

Demand and production of antioxidants are continually shifting from the USA, Western Europe and Japan to the emerging markets mainly China and India. This is a result of the lower wages and environmental regulations which are not as stringent. There are few suppliers that dominate this market and there is great opportunity for expansion. Therefore, it is an object of the present invention to develop and evaluate various combinations of antioxidants in order to determine which combinations are most effective in the treatment of specific conditions. It is a further object of the present invention to use clinical data to determine which combinations of antioxidants consistently yield clinically viable results.

### SUMMARY OF THE INVENTION

According to an embodiment of the present invention, and in the absence of a particular disease treatment protocol and access to a library of reference molecules, a combination of antioxidants is proposed as a supplement based on the best available clinical data.

### DESCRIPTION OF THE INVENTION

As noted above, oxidation is the transfer of electrons (hydrogen) from a substance to an oxidizing agent. The most important of these reactions is Fenton's reaction in which a hydroxyl radical is produced from reduced iron and hydrogen peroxide. An imbalance in the ability of an individual cell to neutralize the oxidative potential will result in damage to cellular components. The presence of such metals in biological systems is essential but also implicated in the level of oxidative stress. Lesser degrees of stress can be easily overcome and the cell can regain its original state, whereas greater degrees of oxidative stress can initiate apoptosis, while intense stresses may cause cell death and tissue necrosis.

The present invention is directed to a combination of antioxidant agents as a potential supplement based on the best available experimental data. The underlying rationale for this combination approach is the fact that in biological systems, there are multiple antioxidant systems and various rate limiting steps. In these processes, for there to be one individual molecule that can support all of these multiple steps in a multitude of enzymatic pathways is highly unlikely. This represents the primary hypothesis for development of an antioxidant regimen using a combination of agents according to the present invention.

The combination of substances proposed according to the present invention does not limit itself to one enzymatic pathway. Rather, it is believed that the administration of a combination of antioxidant agents facilitates the type of general antioxidant environment needed to protect cells from attack by free radicals and/or peroxides. According to another embodiment of the present invention, a low dosage of multiple antioxidant agents is proposed that may reduce the possibility of side effects and avoid a pharmacological response from any single agent

Studies have demonstrated that antioxidants such as beta carotene and Vitamins C, are not beneficial and in many cases harmful as previously stated. Conversely, it has been shown that many free radicals that are apparently neutralized by antioxidants perform valuable functions (i.e., fighting toxins and growth of cancer cells). Accordingly, flooding the body with high dosages of antioxidant agents that completely neutralize free radicals may not be in the best interest of general health. It is therefore believed that the effective administration of antioxidants, in the right combination, will minimize the harmful effects of oxidizing agents such as free radicals, while still allowing them to perform useful functions in the body's natural defense mechanisms.

Scientific research has demonstrated that among men 45 and older, those who got the most vitamins C E, folate and Zinc have fewer DNA-strand breaks in their sperm. (Strand breaks quantify the genetic quality of sperm, which is a characteristic of aging men.) Middle-aged and older men may even have better sperm as a result of increased antioxidants in their diets. However, the findings do not show that antioxidants directly improve sperm quality or increase chances of a healthy pregnancy.

In one study, 80 healthy, non-smoking men between the ages of 22 and 80 were administered up to 700 mg of vitamin C daily, whereas the normal dose is 90 mg (up to 2000 mg is safe by medical standards). According to the study, men aged 45 and up who were administered supplements containing the highest dosages of vitamin C experienced 20% less DNA damage than those who took less of the supplements.

The findings were similar with vitamin E, zinc and folate, although the differences in sperm. DNA damage were smaller. Once again however, older men in the high-intake groups were administered more of each nutrient than is typically recommended, but were still well within the safe ranges. (The recommended daily dosage for vitamin E is 15 mg and no more than 1,000 mg, while for zinc its 11 mg and no more than 40 mg daily. For folate, it's 400 mg and no more than 1,000 mg.)

A study in 2008 by the Cochrane Collaboration, an international consortium of scientists who assess medical research, performed 67 studies with nearly 400,000 participants. The principle goal was to determine if antioxidant supplements reduce mortality in healthy people or those with cardiovascular, neurological, rheumatoid, renal, endocrine, or other diseases. The study suggests that vitamin A, beta-carotene, and vitamin E, in addition to their well-known health benefits, may also increase mortality as previously noted. The underlying reason supplementing with antioxidants may be dangerous is largely a mystery. However, it is believed that high doses of antioxidants may lead to the formation of pro-oxidants stimulating the harmful DNA and cell-damaging reactions they are intended to prevent. Additionally, antioxidants have been shown to interfere with immune-system cells that fight infection and cancer.

The effects of chemotherapy and radiation are being counteracted by antioxidants in medical foods. Animal and aquaculture feeds are growing demand for antioxidants as stated by leading researchers. The top markets in antioxidants are Japan, the US and China with huge increases in sales. This is also true for Latin America, whereby sales in countries such as Mexico, Chile and Brazil are also increasing in this category. The antioxidant market is even expanding to cosmecueticals as in detox skin care and other related products. “The currently popular beauty supplements contain antioxidants such as lycopene, vitamins C and E, hydrolyzed collagen and HA,” said Joosang Park, vice president of scientific affairs, BioCell Technology LLC. “It is expected that there will be product diversification as new products are introduced to target-specific beauty areas, e.g., skin, hair or nails. This trend will be strengthened by scientific substantiation of more ingredients that should be coordinated with a clear communication with consumers.

We believe that manipulating the setup in which the antioxidants are formed can lead to new trends. In addition to producing related components, specialized fruit bars and water bottles can be manufactured. The hope is to extend the use of clinical practice to the next level in the realm of antioxidants.

### Clinical Validity: C-Elegans Experiments

C-elegans is unsegmented, vermiform, and bilaterally symmetrical, with a cuticle integument, four main epidermal cords and a fluid-filled pseudocoelomate cavity. This species has many of the same organ systems as other animals. They feed on bacteria on decaying vegetable matter and have two sexes: hermaphrodites and mates. The anatomy of C. elegans is a mouth, pharynx, intestine, gonad, and collagenous cuticle. Males have a single-lobed gonad, vas deferens, and a tail specialized for mating. C. elegans is transparent and is useful in the study of cellular differentiation and developmental processes in the organism. The patterns of cell lineage are invariant between individuals in contrast to mammals. Wild-type C. elegans hermaphrodite can be stained with the fluorescent dye Texas Red to highlight the nuclei of all cells. C. Elegans is one of the simplest organisms with a nervous system.

The setup for these clinical studies in antioxidant supplements is part of the basis of this invention. Through these experiments we shall be able to better characterize the effects of antioxidants through the testing of different chemical components and concentrations. We plan to develop a whole range of products based on the plan be presented herein. Amongst these products are combinations for age groups, certain conditions and different regions. Therefore, we can also even formulate fruit bars and water supplements based on these experiments.

The scope of this invention applies to any chemical system that involves complexation of the vitamin or molecules that can promote anti-oxidant behavior. This can be extended to metal complexes that can complex with organic molecules and systems to promote this behavior in biological and other systems. One application is in fact the application to vitamins but can be a general chemical tendency.

### Proposed Initial Compounds and Dosages

The formulation of the following compounds and dosages is proposed as an antioxidative treatment regimen within the scope of the claimed invention:
Acyl-L-carnitine—1000 mg
Alpha-Lipoic acid—750 mg
Vitamin C—250 mg
Vitamin D₃—1000 mg
Vitamin E—200 i.u.
Beta carotene—400 i.u.
Curcumin—250 mg
Selenium—300 mg
L-Methionine—1 g
Omega 3—1 g
Ginseng—50 mg

A further useful formulation of the present invention is:
Acyl-L-carnitine—1000 mg
Alpha-Lipoic acid—750 mg
Vitamin C—250 mg
Vitamin D₃—1000 mg
Vitamin E—200 i.u.
Beta carotene—400 i.u.
Curcumin—250 mg
Selenium—300 mg
L-Methionine—1 g
Omega 3—1 g
Ginseng—50 mg
Taurine
Nicotinamide

Taurine is an amino acid and a downstream part of the antioxidant cascade. The first aspect of the invention may include the administration of an amount of taurine. The taurine may be in addition to, or instead of, the supplemented vitamin C described above. Taurine is an unusual amino acid found in a wide variety of animal species. It is thought that taurine protects cellular membranes from toxic components including oxidants. The increase in vitamin taurine levels in an animal diet can contribute to a reduction in free radicals and therefore a reduction in oxidative stress in the animal, in particular in combination with the other components of the invention. The taurine according to the first aspect of the invention may be in any form. It may be powered, crystalline, semi-solid or liquid. The source of the taurine is not limiting. Preferred taurine sources include aminoethylsulfonic acid (C₂H₇NO₃S). Sources may be natural or synthetic. Suitable concentrations of taurine for use according to the present the invention are a preferred level of from about 80 mg/400 kcal, more preferably from about 100, increasing even more preferably from 120, 150, 180, 200, 220, 250, 280, 300, 320, 350, 400 and above in mg/400 kcal diet.

Nicotinamide, also known as niacinamide, NAA, and nicotinic amide, is the amide of nicotinic acid (vitamin B3/niacin), Nicotinamide is a water-soluble vitamin that is part of the Vitamin B complex and is believed to play a in treating diabetes by regenerating islet cells. Nicotinic acid, also known as niacin, is converted to nicotinamide in vivo, and, though the two are identical in their vitamin functions, nicotinamide does not have the same pharmacological and toxic effects of niacin, which occur incidental to niacin's conversion. Thus nicotinamide does not reduce cholesterol or cause flushing, although nicotinamide may be toxic to the liver at doses exceeding 3 g/day for adults. In cells, niacin is incorporated into nicotinamide adenine dinucleotide (NAD) and nicotinamide adenine dinucleotide phosphate (NADP), although the pathways for nicotinic acid amide and nicotinic acid are very similar. NAD+ and NADP+ are coenzymes in a wide variety of enzymatic oxidation-reduction reactions,

Other useful components of the formulation, according to the invention, include, but are not limited to: trace minerals (not direct antioxidants, but function as cofactors within antioxidant metalloenzyme systems); selenium (an essential part of the antioxidantselenoenzyme, glutathione peroxidase); copper, zinc and/or manganese (forming an integral part of the antioxidantmetalloenzymes Cu—Zn-superoxide dismutase and Mn-superoxide dismutase).

In accordance with the method of the first aspect of the invention, the claimed formulation may be administered or consumed, simultaneously, separately, or sequentially. As one of skill in the art will appreciate, in terms of dosage application the claimed formulation could be administered 1-2 times daily, preferably once in the morning and once in the evening. It will be readily understood however, depending on the desired application, that the particular formulation may necessitate a specific component in the mixture for an age group or desired effect. It could be that increasing the amount of one of the components is beneficial for energy levels whereas harmful for others who for example may smoke (certain vitamins are harmful for smokers and benefit others for other secondary effects).

The foregoing description and examples should be considered as illustrative only of the principles of the inventive concept disclosed herein. Exemplary embodiments of the claimed invention may be realized in a variety of formulations and are not intended to be limited by the preferred embodiments described above. Numerous applications of exemplary embodiments will readily occur to those skilled in the art. Therefore, it is not desired to limit the inventive concept to the specific examples disclosed or the exact construction and operation shown and described. Rather, all suitable modifications and equivalents may be resorted to, while still falling within the scope of this application. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention.

The invention claimed is:

1. A composition for administration in a daily dose to male and female humans for promoting specific antioxidant behavior, comprising:
Acyl-L-carnitine—1000 mg; Alpha-Lipoic acid—750 mg; Vitamin C (calcium ascorbate)—250 mg; vitamin C (calcium ascorbate) 250 mg; vitamin D-3 (cholecalciferol) 1000 mg; natural source vitamin E (d-alpha tocopherol/d-alpha tocopheryl acid succinate) 200 I.U.; beta-carotene (from natural d. salina) 400 I.U.; curcumin—250 mg; selenium (l-seleno methionine)—300 mg; L-Methionine—1 g; Omega 3—1 g; ginseng—50 mg.

2. The composition of claim 1, further comprising nicotinamide (niacinamide ascorbate) in the amount of 15 mg.

3. The composition of claim 1, further comprising taurine in the amount of 80 mg.

4. The composition of claim 1, further comprising one or more organic or bio-compatible salts to control oxidation.

5. The composition of claim 4, wherein the bio-compatible salt is bicarbonate.

6. The composition of claim 1, further comprising trace minerals to control oxidation.

7. The composition of claim 6, wherein the trace minerals are selected from the group consisting of copper, zinc and/or manganese.

8. A method of administering in a daily dose to male and female humans for promoting specific antioxidant behavior, a composition comprising:
Acyl-L-carnitine—1000 mg; Alpha-Lipoic acid—750 mg; Vitamin C (calcium ascorbate)—250 mg; vitamin C (calcium ascorbate) 250 mg; vitamin D-3 (cholecalciferol) 1000 mg; natural source vitamin E (d-alpha tocopherol/d-alpha tocopheryl acid succinate) 200 I.U.; beta-carotene (from natural d. salina) 400 I.U.; curcumin—250 mg; selenium (l-seleno methionine)—300 mg; L-Methionine—1 g; Omega 3—1 g; ginseng—50 mg.

9. The method of claim 8, the composition further comprising nicotinamide (niacinamide ascorbate) in the amount of 15 mg.

10. The method of claim 8, the composition further comprising taurine in the amount of 80 mg.

11. The method of claim 8, the composition further comprising one or more organic or bio-compatible salts to control oxidation.

12. The method of claim 11, wherein the bio-compatible salt is bicarbonate.

13. The method of claim 8, the composition further comprising trace minerals to control oxidation.

14. The method of claim 13, wherein the trace minerals are selected from the group consisting of copper, zinc and/or manganese.

15. The method of claim 8, further comprising the step of using salt dissociation inside nanotubes (carbon or boron-nitride) to promote anti-oxidative activity.

16. The method of claim 8, further comprising the step of using reactions inside nanotubes (carbon or boron-nitride) to promote anti-oxidative activity.

17. The method of claim 8, further comprising the step of using industrial productions using metallic interactions with nanotubes (carbon or boron-nitride) to promote anti-oxidative activity.

18. The method of claim 8, further comprising the step of using metal-nanomaterial complexes to improve interaction with substances promoting oxidation.

19. The method of claim 8, further comprising the step of using of metal-nanomaterial complexes to improve interaction with substances promoting oxidation.
